(19) United States
(12) Patent Application Publication
(10) Pub. No.: US 2021/0299289 A1
(43) Pub. Date: Sep. 30, 2021

(54) METHODS AND APPARATUS FOR VOLUMETRIC INACTIVATION OF VIRUSES BY ACOUSTIC RESONANCE STIMULATION USING NON-IONIZING GIGAHERTZ ELECTROMAGNETIC RADIATION

(71) Applicants: Luke Christopher Raymond, Redwood City, CA (US); Julie Patricia Saiki, Redwood City, CA (US); John Paul Christie Collman, Redwood City, CA (US); Johan Oscar Lennart Andreasson, Redwood City, CA (US)

(72) Inventors: Luke Christopher Raymond, Redwood City, CA (US); Julie Patricia Saiki, Redwood City, CA (US); John Paul Christie Collman, Redwood City, CA (US); Johan Oscar Lennart Andreasson, Redwood City, CA (US)

(21) Appl. No.: 17/216,648

(22) Filed: Mar. 29, 2021

Related U.S. Application Data
(60) Provisional application No. 63/001,041, filed on Mar. 27, 2020.

(51) Int. Cl. A61L 2/00

(52) U.S. Cl. CPC A61L 2/0029; A61L 2/24

(57) ABSTRACT

According to some aspects, there is provided a method of selectively inactivating viruses through acoustic resonance by non-ionizing gigahertz electromagnetic radiation. In some implementations, there is provided an apparatus comprising a power processing unit; a microwave source; an antenna; a control unit; wherein the spatial and temporal characteristics of the electromagnetic field are optimized to maximize viral inactivation at the target location while minimizing overall radiation exposure. Methods of delivering high peak energy into the virus structure with minimal total average power are also provided.

### CROSS-REFERENCE TO RELATED APPLICATIONS

This application claims the benefit of U.S. provisional patent application Ser. No. 63/001,041, filed on Mar. 27, 2020, and entitled “METHODS AND APPARATUS FOR VOLUMETRIC INACTIVATION OF VIRUSES BY ACOUSTIC RESONANCE STIMULATION USING NON-IONIZING GIGAHERTZ ELECTROMAGNETIC RADIATION,” which is incorporated by reference herein in its entirety.

### TECHNICAL FIELD

The subject matter described herein relates to methods of selectively inactivating viruses by acoustic resonance stimulation using non-ionizing gigahertz electromagnetic radiation, to methods of reducing viral transmission, to methods of treatment and prevention, and to apparatuses designed to achieve these methods.

### BACKGROUND

The COVID-19 pandemic, caused by the SARS-CoV-2 virus, has highlighted the ability of a major viral pandemic to cause global crisis, even in modern times. The major challenges in containing the spread of viruses such as SARS-CoV-2 are their person-to-person transmissibility via respiratory droplets as well as their relatively high aerosol and surface stability (Van Doremalen N, Bushmaker, et al.). Vaccines, while effective, are generally virus specific and can take years to develop and distribute on a global scale.

More so than at any previous time in modern history, there is an urgent need to develop methods of rapidly and efficiently decontaminating public and private spaces to reduce the likelihood of another COVID-19 scale pandemic. Current methods of disinfection which include chemical disinfectants and ultraviolet light are labor-intensive and not safe for direct human contact. There is currently no widely-available, cost-effective method to inactivate viruses in spaces where humans are also present.

The significant time required to develop pharmaceutical agents, such as drugs or vaccines, introduces a long waiting period between the outbreak of a new disease and the approval of the treatments, if these can be developed. During this period, in particular, there is a large need for broad-spectrum antiviral methods that do not rely on chemical interactions such as vaccines, small-molecule drugs or antibodies. Being able to quickly target new viruses, including those with unknown surface structure, with a flexible device that works in a range of settings would be highly beneficial. A non-chemical, non-thermal method, without side effects, that can inactivate several of existing and new viruses would fill a significant unmet need. This is particularly the case for pathogenic viruses that cause infectious disease in humans, but it is also applicable to animals, livestock, and plants. By relying on a physical method, viruses that are transmitted through the air or in droplets, or on surfaces, can be inactivated. The development also does not require large and costly clinical trials.

There is an ongoing unmet need for novel innovations to treat patients infected with viral pathogens in order to reduce the severity of the disease and secondary complications including progression to pneumonia, acute respiratory distress syndrome, organ injury, septic shock, or death.

### SUMMARY

The method described herein comprises use of non-ionizing electromagnetic radiation at levels safe for humans for the inactivation of different kinds of viruses. This method can be used to inactivate viruses quickly and efficiently in air, aerosols, liquids, solids or tissues, or on surfaces, by non-thermal means, and to non-invasively reduce viral load in living tissues for the treatment of disease.

Specific material properties of a virus, primarily the shape, physical size, and rigidity confer acoustic vibrational modes in the GHz range. Electromagnetic radiation with a frequency within the resonance peak stimulates this mode and amplifies the oscillations in the virus structure. A displacement force is achieved by the opposite charge density between the core, which has a high concentration of negatively charged nucleic acids, and the outer layers, including the capsid and envelope, which are positively charged. When tensile limits are exceeded, the virus is damaged by fracturing.

There is a minimum power level required for the microwave radiation to achieve a meaningful acoustic resonance, and previous work (Yang et al.) has demonstrated that the time necessary to inactivate a specific percentage of viruses within a sample goes down as field intensity is increases. However, Yang et al. also demonstrated that a continuous wave (CW) microwave source requires almost 1 kW/m² to inactivate nearly 100% of H3N2 influenza viruses in a sample exposed near its acoustic resonant frequency of ˜8 GHz for 14 min. Such levels of radiation exposure exceed safe limits prescribed by the FCC and other regulatory agencies. Because the danger to humans is primarily related to thermal heating of exposed tissue, some implementations described herein are capable of inactivating viruses through repeated exposure of extremely high intensity, but relatively short duration pulses, while staying well within the safe limits of human exposure.

In some implementations, there is provided an apparatus comprising a power processing unit; a microwave source; an antenna; a control unit; wherein the spatial and temporal characteristics of the electromagnetic field are optimized to maximize viral inactivation while minimizing overall radiation exposure at the target location. In some variations, the methods of coupling high peak energy into the virus structure with minimal total average power are also detailed.

By keeping the electromagnetic radiation intensity within safe human limits, it is also possible to implement the invention as a therapeutic for reducing viral load within patients and as a device for treating bodily fluids or tissues.

Since the peak intensity of the electromagnetic waves is much higher than in a CW approach, the distance over which the device can produce the necessary field strength for viral inactivation is much larger, exceeding 100s of meters when coupled with directional and/or rotating antennas as detailed herein, allowing for sterilization of large indoor and outdoor spaces.

At the heart of an example embodiment of the invention is a controller that is capable of modulating the electromagnetic radiation temporally and/or spatially to optimize the inactivation properties of the device. In some variations the controller determines pulse width, power, repetition rate, carrier frequency, and direction of emission. The controller may also send and receive data through a network and/or user interface, and coordinate or control a plurality of devices.

While some implementations are intended to be mounted permanently indoors or outdoors, others are small enough to be portable and handheld, still others are capable of being mounted to a vehicle or robot.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to web application user interfaces, it should be readily understood that such features are not intended to be limiting. The claims that follow this disclosure are intended to define the scope of the protected subject matter.

### DESCRIPTION OF DRAWINGS

The patent or application file contains at least one drawing executed in color. Copies of this patent or patent application publication with color drawing(s) will be provided by the Office upon request and payment of the necessary fee. The accompanying drawings, which are incorporated in and constitute a part of this specification, show certain aspects of the subject matter disclosed herein and, together with the description, help explain some of the principles associated with the disclosed implementations. In the drawings,

FIG. 1 depicts an example embodiment of the invention detailing various components, in accordance with some example embodiments;

FIG. 2 depicts an example electromagnetic wave profile, in accordance with some example embodiments;

FIG. 3 depicts an example embodiment that allows for millisecond pulses that we developed, in accordance with some example embodiments;

FIG. 4 depicts an example embodiment with slotted antenna, in accordance with some example embodiments;

FIG. 5 depicts an example electromagnetic wave profile, in accordance with some example embodiments;

FIG. 6 depicts an example electromagnetic wave profile, in accordance with some example embodiments;

FIG. 7 depicts an example electromagnetic wave profile, in accordance with some example embodiments;

FIG. 8 depicts an example electromagnetic wave profile, in accordance with some example embodiments;

FIG. 9 depicts an example electromagnetic wave profile, in accordance with some example embodiments;

FIG. 10 depicts gain pattern for horn antenna used in the embodiment device shown in FIG. 3, in accordance with some example embodiments;

FIG. 11 depicts an example embodiment of a rotating antenna placed in the ceiling of a room in a public space, the public space being hospital hallway, in accordance with some example embodiments;

FIG. 12 depicts an example embodiment of a rotating antenna placed in the ceiling of a room in a public space, public space being grocery store, in accordance with some example embodiments;

FIG. 13 depicts an example embodiment of a rotating antenna placed in the ceiling of a room in a public space, public space being an airport terminal, in accordance with some example embodiments;

FIG. 14 depicts the embodiment of the waveform for the device shown in FIG. 3, in accordance with some example embodiments;

When practical, similar reference numbers denote similar structures, features, or elements.

### DETAILED DESCRIPTION

The following aspects and embodiments thereof described and illustrated below are meant to be exemplary and illustrative, not limiting in scope.

Aspects of the disclosure may be embodied as a system, method or program code/instructions stored in one or more machine-readable media. Aspects may take the form of hardware, software (including firmware, resident software, micro-code, etc.), or a combination of software and hardware aspects that may all generally be referred to herein as a “circuit,” “circuitry,” “module,” or “system.” The functionality presented as individual modules/units in the example illustrations can be organized differently in accordance with any one of platform (operating system and/or hardware), application ecosystem, interfaces, programmer preferences, programming language, administrator preferences, etc.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the disclosure. In general, structures and functionality presented as separate components in the example configurations may be implemented as a combined structure or component. Similarly, structures and functionality presented as a single component may be implemented as separate components. These and other variations, modifications, additions, and improvements may fall within the scope of the disclosure.

The following aspects and embodiments describe a series of systems, methods, techniques, and logical flows that illustrate various embodiments of a power generator, control unit or units, and an electromagnetic radiation source or sources in the GHz range tuned specifically to inactivate viruses through acoustic resonance.

The method described herein comprises use of non-ionizing electromagnetic radiation for the inactivation of different kinds of viruses. This method can be used to inactivate viruses quickly and efficiently in air, aerosols, liquids, solids or tissues, or on surfaces, by non-thermal means, and to non-invasively reduce viral load in living tissues for the treatment of disease. An apparatus to implement this method is also described in several embodiments.

The mechanism of viral inactivation uses a structure resonance in the virus that is excited by electromagnetic (EM) energy. The resonant microwave absorption of viruses is well known, and the absorption of energy and the resulting inactivation of the virions have been described in published work (Yang, S.-C. et al. Efficient Structure Resonance Energy Transfer from Microwaves to Confined Acoustic Vibrations in Viruses. Scientific Reports 5, (2015); Sun, C.-K. et al., Resonant Dipolar Coupling of Microwaves with Confined Acoustic Vibrations in a Rod-shaped Virus. Scientific Reports 7, (2017); Liu, T.-M., et al., Microwave resonant absorption of viruses through dipolar coupling with confined acoustic vibrations, Appl. Phys. Lett. 94, 043902 (2009); Sun, C.-K. Liu, T.-M., and Chen, H.-P., Detect and identify virus by the microwave absorption spectroscopy, US patent US20090237067A1; Sun, C.-K. and Liu, T.-M., Microwave resonant absorption method and device for viruses inactivation, US patent application US20110070624A1). The effect has been termed microwave resonant absorption (MRA) or Structure Resonance Energy Transfer (SRET). Here we refer to it as acoustic resonance or just resonance.

The material properties of the virus, primarily the shape, physical size, and rigidity confer acoustic vibrational modes in the GHz range. Electromagnetic radiation with a frequency within the resonance peak stimulates this mode and amplifies the oscillations in the virus structure. The displacement force is achieved by the opposite charge density between the core, which has a high concentration of negatively charged nucleic acids, and the outer layers, including the capsid and envelope, which are positively charged. When tensile limits are exceeded, the virus is damaged by fracturing. As an example, the resonant frequency of the H3N2 virus is approximately 8.4 GHz (Yang, et al., 2015).

Similar research into mechanical resonance has shown acoustic modes for even the smallest viruses (Burkhartsmeyer, et al., Optical Trapping, Sizing, and Probing Acoustic Modes of a Small Virus, Appl. Sci. 2020, 10(1), 394) and femtosecond lasers have similarly been used to vibrate and fracture viruses in published work by researchers at Arizona State University (Tsen, K. T., et al. Studies of inactivation of encephalomyocarditis virus, M13 bacteriophage, and Salmonella typhimurium by using a visible femtosecond laser: insight into the possible inactivation mechanisms. Journal of Biomedical Optics 16, 078003 (2011)).

The resonance is accurately described by mechanical models of the virus that incorporate the known physical variables of the virus and the charge distribution. Experimental work, including the references above, have validated the corresponding microwave absorption peaks. Also, Yang, et al., for example, demonstrated a strong antiviral effect after exposure to microwaves, as measured by a viral replication assay using an influenza virus.

Ultimately, the effect is similar to a wine glass shattering when an opera singer hits the right frequency at sufficient volume. When the viral envelope has been damaged through resonance, the ability to invade host cells and replicate is greatly reduced. The RNA core may still be largely intact initially, but exposed viral RNA is unstable and will decay quickly without the protective envelope. In vivo, exposed RNA is susceptible to RNases and other mechanisms of degradation. This mechanically destructive method of viral inactivation is analogous to the outcome of using detergent to disrupt the lipid envelope, which is well-documented as a very effective means of reducing viral infectivity.

The resonant excitation acts on the dipolar mode of the confined acoustic vibrations (CAVs) of the virus (Yang, et al.). It is a mechanical effect that depends on the morphology of the virion and its size. For a spherical virus, the fundamental and first harmonic frequencies are known to apply and a smaller virus with the same rigidity will therefore exhibit a higher frequency. For rod-shaped viruses, the confined acoustic modes are the n=4N−2 longitudinal modes, as described by Sun, et al., These depend on both the length and diameter of the virion and provide a range of resonant frequencies that can potentially be used for the purposes of viral inactivation. For viruses with more irregular shape, such as the Ebola virus, resonances are difficult to calculate but may still be present such that these viruses may still be inactivated microwaves at a specific resonant frequency.

The center frequency of the absorption peak depends on the size and rigidity of the virus. The width of the resonance peak (in GHz) is also determined by the structural features of the virus, including the center frequency, how rigid the capsid and/or envelope is, and how homogeneous the distribution of viral particles is within a sample. For reference, Yang et al. measured a center frequency of 8.2 GHz and a full width at half maximum (FWHM) of 4.2 GHz for the spherical (˜100 nm diameter) H3N2 influenza virus. In prior publication, the group of Prof. Sun demonstrated a frequency of 42 GHz for the 29.5 nm diameter Perina nuda virus (PnV) (Sun, Liu, and Chen), ˜43 and 40 GHz for the 28.5 nm diameter Enterovirus 71 (Sun, Liu, and Chen; Sun and Liu). The same group (Sun et al.) found peaks at 6.6 GHz, 21.6 GHz, 35.7 GHz, and 47.8 GHz for the white spot syndrome virus (WSSV), a rod-shaped virus of length ˜300 nm and diameter ˜100 nm. Other experimental validations of GHz absorption of viruses include 60 GHz peaks for the Tobacco Mosaic Virus (TMV) (Karpova, O. V. et al., Stimulated low-frequency Raman scattering in tobacco mosaic virus suspension, Laser Phys. Lett. 13 085701 (2016)) and 32 GHz for the 25-nm PhiX174 virus (Burkhartsmeyer, et al.),

The systems, methods, techniques, and logical flows herein are universally applicable to most of the more than 131 characterized human viruses, as well as uncharacterized and novel viruses involved in human health and disease. They are also applicable to viruses affecting animals, including livestock, pets, and wildlife. The method and apparatus described herein therefore applies to, but is not limited to, the following viruses: Adeno-associated virus, Aichi virus, Australian bat lyssavirus, BK polyomavirus, Banna virus, Barmah forest virus, Bundibugyo virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Cuevavirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, Filoviridae, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O′nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Sudan virus, Tai Forest, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and/or Zika virus.

There is a minimum power level required for the microwave radiation to achieve a meaningful acoustic resonance. Yang et al. demonstrated that a continuous wave (CW) microwave source requires almost 1 kW/m² to inactivate nearly 100% of H3N2 influenza viruses in a sample exposed near its acoustic resonant frequency of ˜8 GHz for 14 min. The calculations estimated an inactivation threshold field intensity of 86.9 V/m, corresponding to an average microwave power density of 82.3 W/m², and at this level the inactivation ratio was close to 40%.

There are several regulatory limits for microwave emissions. The International Commission on Non-Ionizing Radiation Protection (ICNIRP) human exposure threshold is

$$7.2 * [ 0.05 + 0.95 * {( \frac{t}{360} )}^{0.5} ]$$

kJ/m² is applicable to short-duration pulses. The FCC limit specified by 47 CFR § 1.1310 is set to time average powers of 5 mW/cm² (50 W/m²) for occupational/controlled exposure with an averaging time less than 6 minutes and 1 mW/cm² (10 W/m²) for general population/uncontrolled exposure with a averaging time less than 30 minutes. According to Yang et al., the IEEE Microwave Safety Standard specifies that the spatial averaged value of the power density in air in open public space shall not exceed the equivalent power density of 100(f/3)^{1/5} W/m² at frequencies between 3 and 96 GHz (f is in GHz). At 8 GHz, this corresponds to ˜122 W/m².

In summary, continuous human exposure to microwaves in the GHz frequency range at levels of 100 W/m² to several kW/m² is incompatible with existing regulations. This requires an effective antiviral apparatus to work in environments without direct human exposure as long as the emission is continuous, or for the regulations to be changed. This allows for targeted approaches where the direction of the emission or human protective gear prevents the direct exposure the operator to the microwave emission, or where objects are exposed without a direct human operator, for example on a conveyor belt or an autonomous robot.

A better method, which we propose herein, is a controlled microwave emission that uses spatiotemporally modulated emission profile to deliver high instantaneous field strengths over a short time interval and/or in a well-defined spatial direction or location. At the heart of the method is achieving extremely high peak powers and fields, while minimizing heating of the targeted area or volume. The method is further improved by optimizing the emission frequency, power envelope, combinatorial use of multiple emitters, and/or the use of scheduling, sensor data, machine learning, and other intelligent approaches to minimize unwanted exposure while maximizing the anti-viral effect. We also describe embodiments of devices that achieve these goals.

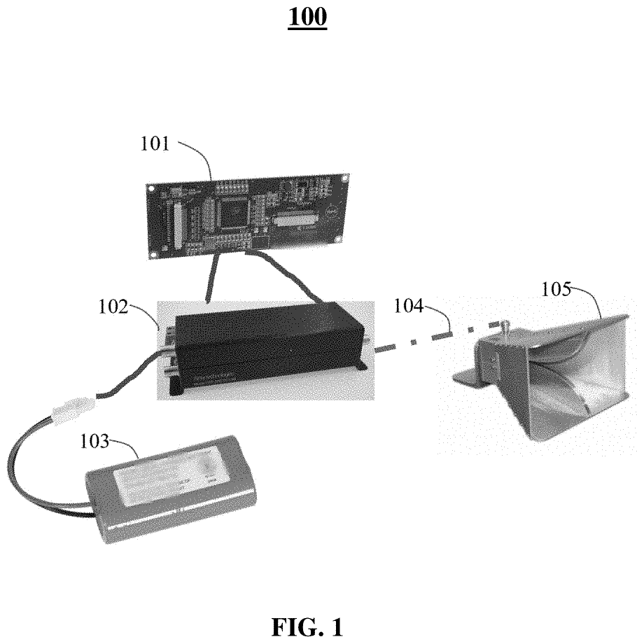

In short, the device or apparatus with the components for practicing the consists of a power processing unit that converts the input power into power for microwave generation, one or more microwave sources, one or more emitters or antennae, and a control system and a control system that ensures that the emission is not constant in space and time. Various embodiments of such an apparatus are given below and FIG. 1 illustrates a basic such device or apparatus. In FIG. 1, the power source 103 is a battery but more commonly it is the electrical grid. The power connection links it to a combined power processing unit and microwave source 102. The microwave power is channeled via a waveguide 104 to a microwave antenna 105, which in this example is a horn antenna. The entire operation is controlled by the control unit 101 which controls the power processing, the output power of the microwave source, e.g., a magnetron, and the pulsing of the power and scanning of the antenna.

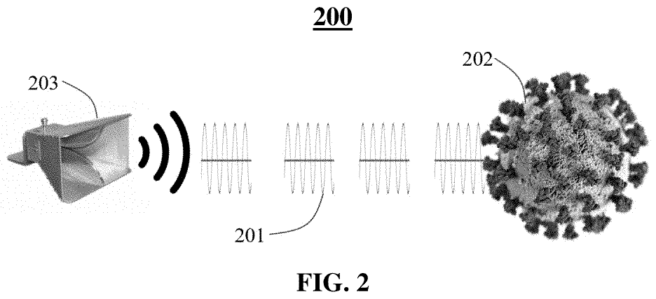

By pulsing the microwave emission, in microsecond-to-millisecond time intervals, the peak power can be very high while the average emission is kept well within prescribed regulatory limits. For example, a duty cycle of 0.1% allows for a 10 kW/m² peak power to be delivered for a long time (within FCC limits), which is much higher than the ˜1 kW/m² threshold for almost full inactivation reported by Yang et al. FIG. 2. illustrates a pulsed waveform 201 emitted from the device 203, delivering microwave energy at a frequency close to the resonant frequency of the virus 202.

For a basic on-off pulse profile, the microwave power is turned on and off at short time scales. The minimum pulse width is longer than several cycles, i.e., at least one nanosecond. In practice the pulses should be sufficiently long to fully excite all virions in an exposed sample and sustain the power delivery until they are inactivated. This time scale may be microseconds (˜0.1 μs, ˜0.3 μs, ˜1 μs, ˜3 μs, ˜10 μs, ˜30 μs, ˜100 μs, ˜300 μs, and the like) to milliseconds (˜1 ms, ˜3 ms, ˜10 ms, ˜30 ms, ˜100 ms and the like). A system that can deliver very high peak powers is beneficial since it allows for the virions to experience a force larger than the inactivation threshold.

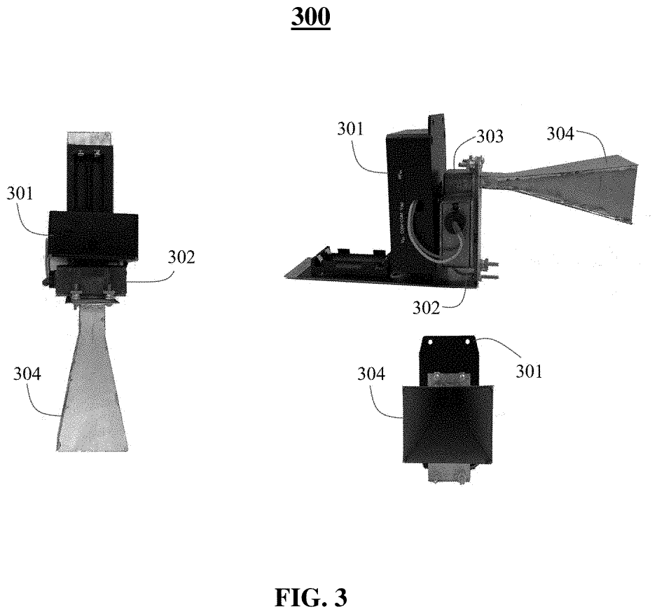

In FIG. 3. we show a system that we developed that allows for millisecond pulses, i.e., quasi-continuous conditions for a virion and orders of magnitudes longer than traditional pulsed systems. It consists of combined power processing unit and control unit 301 which delivers high voltage pulses to a magnetron 302. The output waveguide of the magnetron 303 guides the radiation to a horn antenna 304.

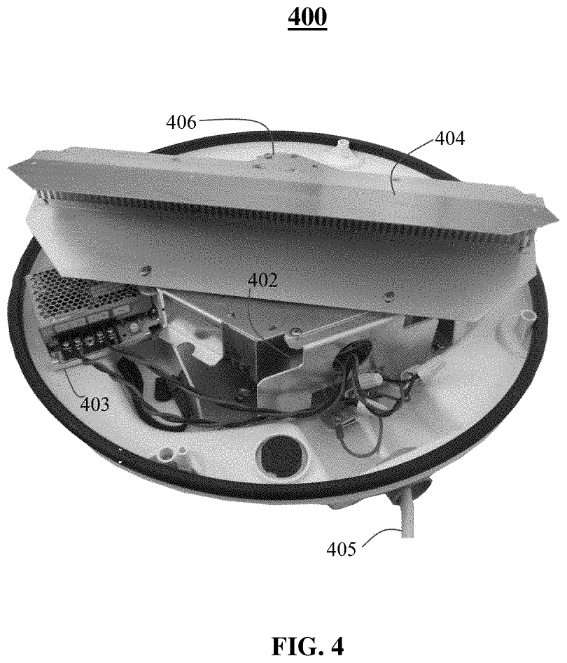

FIG. 4. Shows a second system where a power processing unit 403 is connected to the grid via a cable 405 and connected to a magnetron 402 and control unit (not visible). The output of the magnetron is guided to a slotted antenna 404 which rotates around the attachment point 406 to continuously scan the beam in a circle to emit radiation that is focused in the vertical direction and omnidirectional in the azimuthal direction, on average.

The pulsing duty cycle can be anything greater than 0%. For low average power, a low duty cycle is preferred. Example duty cycles are 10%, 1%, 0,1%, 0.01%, 0.001%, 0.0001% and 0.00001% but the duty cycle is not limited to these values and can be set to any number in between, or a larger or a smaller number.

The duty cycle does not have to be single value. For instance, pulse start spacing may remain the same while pulse width varies, or pulse width may remain the same while spacing varies, or subsequent pulse spacings should be smaller instead of larger. The “trains” could be assembled into repeating pulsed patterns to maximize viral inactivation for a given target area.

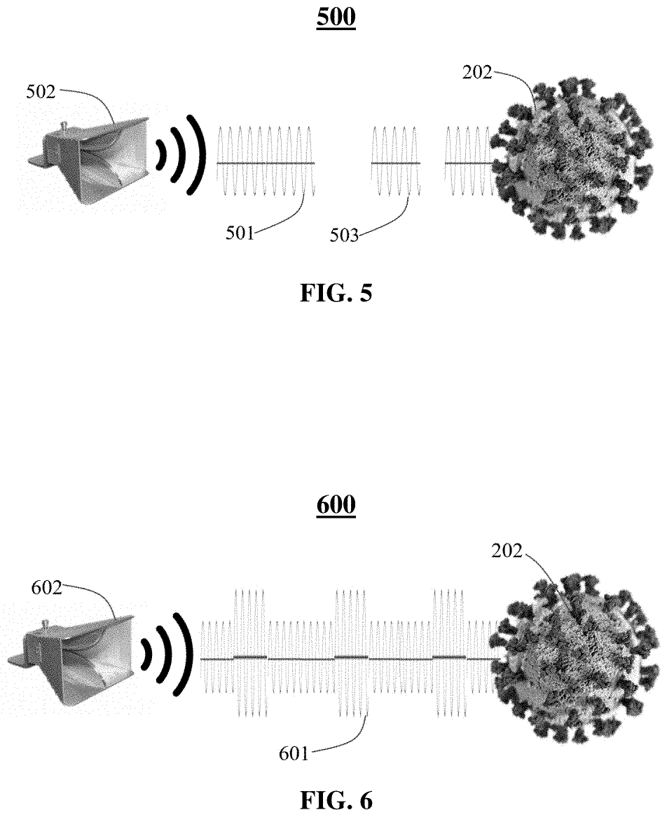

FIG. 5. shows one example where a device 502 emits pulses of long durations 501 and short durations 503 toward a virus 202.

If beneficial in the application, the microwaves can be emitted in bursts, wherein several short pulses are generated followed by a longer pause, or each pulse can have a different length. Increasing or decreasing pulse widths are possible, as are alternating pulse widths. The important aspect is that the system has one or multiple levels of pulse width control and that it can be set for optimal performance (antiviral effect), either permanently or dynamically by an algorithm or an operator.

Further control of the pulses is attained through control of the power in each pulse. The power may be constant, ramped up (chirped), ramped down, or be set to an arbitrary waveform (on a longer time scale than the fundamental microwave time period). Setting the arbitrary waveform may include alternating between two or more power levels (multi-level pulsing). FIG. 6 illustrates such a beam 601 with time-varying intensity being emitted from a single emitter 602 toward a virus that is being inactivated 202. Multilevel pulsing or continuously variable power may improve the viral inactivation for an overall delivered power and is not limited to a single source or frequency.

As in the pulse width case, controlling the power envelope to optimize for performance is a key aspect.

In addition to, or in lieu of, emitting many pulses, the device can be optimized for maximum peak power performance, in which a few cycles (1, 10, 100, 1000, or a number in between) of radiation is emitted in one or a few pulses at very high powers, similar to an electromagnetic pulse (EMP). This mode may be used for viruses for which the efficacy increases with higher peak power. There is no clear demarcation between this peak power mode and the pulsing mode above since the tradeoff between peak power, average power, pulse length, and pulser shape will be determined by the designer and/or operator for the specific application. For this reason, a flexible device that allows for algorithmic, manual, or feedback control of the emission sequence with respect to these variables may be valuable.

Different strains of viruses have different resonant frequencies and homogeneity. This is addressed by a device that has a variable frequency emission or multiple emitters, each with a different frequency. Controlling the frequency, in combination with average powers and pulse length, spacing, and power envelope is important for performance. One example is a pulse train where in each pulse has a different frequency. A second example is a frequency sweep within each pulse. A third example is alternating between two or more different frequencies in a repetitive temporal emission pattern. A fourth example is tuning the power for each of the emitted frequencies as the max allowable emitted power is frequency dependent. A fifth example is emitting a first frequency for one portion of each pulse followed by a second frequency for the remainder of the pulse, to account for changes in resonant frequency due to morphological changes as the virions are excited but not fully inactivated. The above examples are by no means limiting and in practice there are an infinite number of ways to combine variable frequency, power, and pulsing characteristics. In one embodiment, the device described herein allows for algorithmic, manual, or feedback control of frequency.

There are viral inactivation benefits to be gained by using pulse trains, or pulses of differing widths and electromagnetic wave frequencies in a specific sequence and timing, to maximize the resonant excitations of the virus shell. FIG. 7 illustrates such an example. In FIG. 7, a device 703 emits two synchronized pulse trains, one with a low microwave frequency 701 and one with a high microwave frequency 702, toward a virus 202. In this particular example, the pulse widths and the magnitudes are identical but they can also be different and the pulse trains may be asynchronous.

As the virus shell moves from a non-energized state via acoustic resonance, the rate of vibration or oscillation in the outer layers (capsid and/or envelope) may change over time due to mechanical deformations during the duration of the pulse. Each pulse causes the virus shell to vibrate on an axis about the equator of the shell and incur damage. Due to the elasticity of the shell, it will rebound to its resting state after the pulse, but initial damages may remain and subsequent pulses will add to the damage until the virus breaks. Each subsequent pulse may have a different width or frequency to optimize the inactivation of the virus as it changes. However, a single large pulse may be sufficient and, in some cases, ideal.

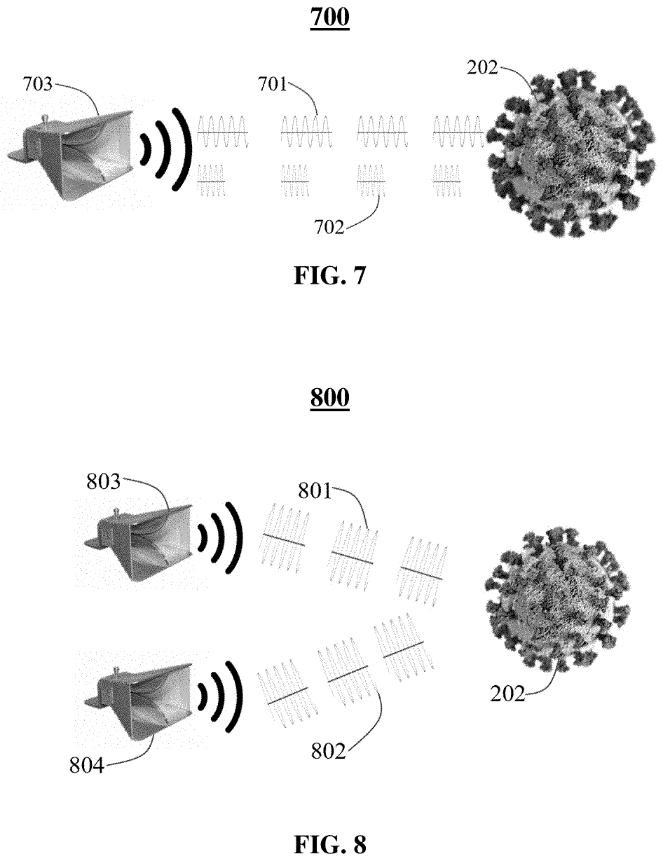

Using multiple emitters with the same frequency or different frequency is another method for improving performance. In FIG. 8, two synchronized pulse trains, 801 and 802, are emitted from two separate emitters, 803 and 804, such that they constructively interfere at the location of the virus, 202. This leads to higher intensity at the targeted area but lower exposure elsewhere.

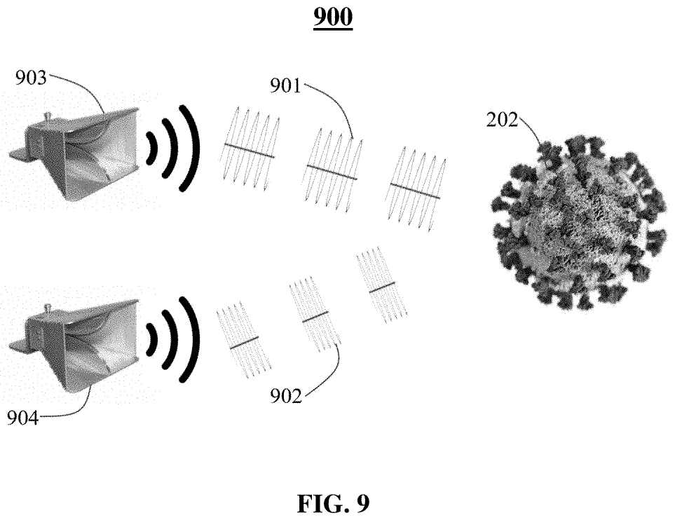

FIG. 9. illustrates an example where two emitters, 903 and 904, radiate pulse trains at two different frequencies, 901 and 902, to excite more than one resonance within the virion. In the case where the second frequency is a multiple of the first, constructive interference may improve the performance at the location of the virus 202.

Using two separate frequencies may be particularly useful in the case when the second frequency is twice the primary resonant frequency or, as in the case of rod-shaped viruses, a multiple of a fundamental frequency N (n=4N−2). There exist second-order resonant harmonics that can be leveraged to increase the total amplitude experienced at the outer layers of the virus. There are many viruses that are not spherical, such as variola, which is a complex poxvirus, or the tobacco mosaic virus, which is helical or rod-shaped, or poliovirus, which is icosahedral. For non-spherical viruses such as Ebola, the combination of harmonically unrelated frequencies can be used to create a combinatorial effect of power and frequency that uses the specific shape of the capsid to absorb sufficient resonant energy such that the stress threshold of the layers is reached.

Concentrating the emission and directing it to the target area or volume may in many instances be critical for improving performance and minimizing off-target exposure. For this, a device will use one or several (a plurality) or elements to direct the electromagnetic field. The element may be an antenna, a waveguide, a phase array, a transmission line, a movable element such as a gimbal, a reflective surface, a parabolic dish, a dish of a different geometry, a symmetric parabola, a spoiled parabola, a slot antenna, a horn antenna, or a combination thereof. Any item used to transmit or radiate microwave can be used and the above list is seen as an example that is not exhaustive. For the purposes of the descriptions below, the word antenna will be used with the understanding that any of the above elements, or any combination thereof can be used in its place.

The antenna may concentrate the electromagnetic field in a certain direction, compared with an omnidirectional emitter. Any useful microwave system will have an emitter, but the device described herein optimizes the emission profile.

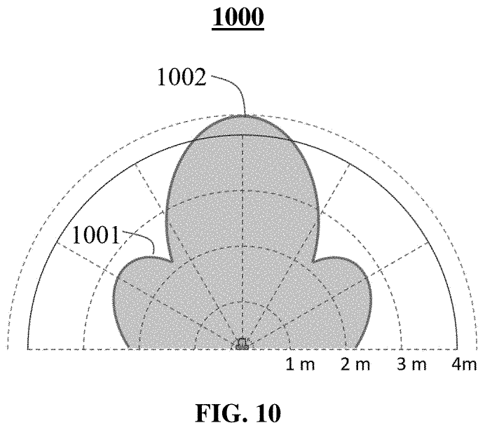

FIG. 10. shows the gain pattern 1001 for the 15 dB horn antenna 304 used in one embodiment of our device (see FIG. 3). The gain increases the field strength in the direction of the antenna output 1002 and therefore increases the resonant absorption accordingly along with the antiviral effect, for a given total power. The distance over which the antiviral effect is meaningful similarly increases with peak power and the gain of the antenna. Side lobes 1001 are present in this particular case but very little intensity is radiated in the opposite direction of the antenna output.

A static antenna allows for a specific target area or volume to be irradiated. The device can also deliver electromagnetic radiation omnidirectionally from a single emitter, either by scanning a directional emitter or by using a non-directional antenna. For applications where the emission should cover the complete volume or area, or at a minimum a larger subset of volume or area than the width of the beam can cover, several methods can be employed to scan the beam. In one embodiment that we have implemented, an antenna is placed on a rotating mount, thereby sweeping a narrow beam around a complete 360-degree turn in a repeating fashion. Such an antenna can be a horn antenna, a slot antenna (see FIG. 4.), or a parabolic dish that focusses the beam tightly in the azimuthal direction. The vertical direction can be tuned to a wide or narrow focus, depending on the application. The directional radiation beam can be mounted to gimbal controlled by a computer or otherwise, whereby the scanning speed is adjusted to the spread of the beam to fully illuminate the desired target area.

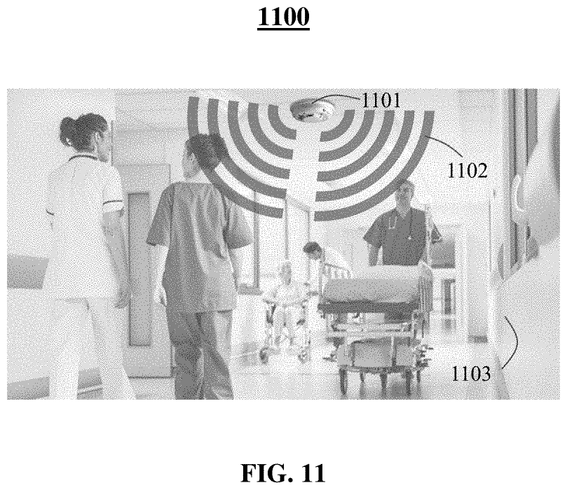

FIG. 11 illustrates one such embodiment where a rotating antenna is placed in the ceiling of a room in a public space to continuously inactivate virions in the air, in aerosols, and on surfaces, thereby lowering the risk of viral transmission. In FIG. 11, the shape of the device 1101 is a round disc mounted in the ceiling of a hospital hallway 1103. By using a spinning antenna inside of the device, and rotationally symmetric pattern 1102 is emitted to inactivate viruses across the entire open area.

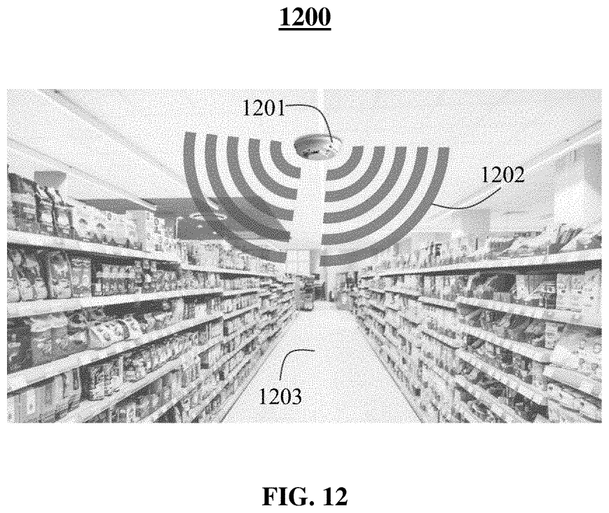

FIG. 12. shows a second example of a ceiling mounted device 1201 in a public space. In this case, the building is a grocery store 1203 that is continuously disinfected by means of microwaves 1202 filling the volume. The average power of the microwaves is low but the scanning of the beam together with the high peak powers ensures efficient volumetric viral inactivation on the time scale of seconds or minutes.

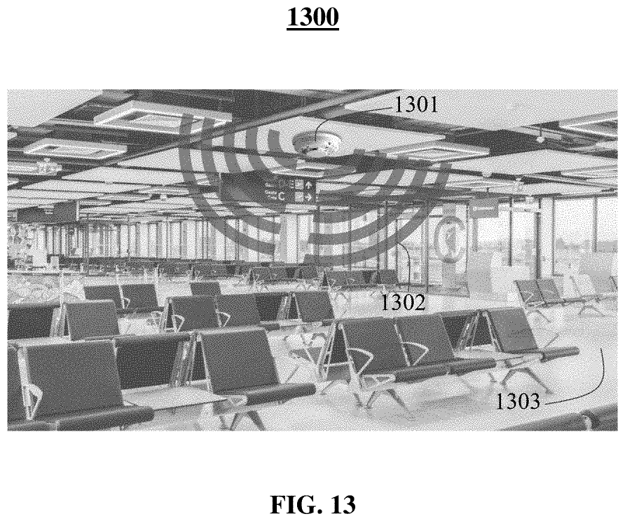

FIG. 13 shows a third example. In this case, the building is an airport terminal 1303. As before, the radiation 1302 is emitted from a ceiling mounted device 1301. However, the device can also be mounted on a wall, on the floor, on a separate stand, or inside the ceiling, as long as the ceiling material does not markedly absorb the microwaves.

Higher peak powers or better spatial control can be achieved by placing the antenna on holder that can move in two dimensions (vertical and azimuthal, for example). The scanning can be performed in a repeated fashion, or based on sensor data, scheduling, algorithms, feedback, or manual control.

In one embodiment, several emitters or antennae are incorporated. They can be used for emitting microwaves with different wavelengths, as described above, but also for spatial control and minimization of exposure to non-targeted regions.

FIG. 8 illustrates that multiple beams from a plurality of emitter arrays can be used to create constructive interference increasing the viral inactivation energy for a targeted region while minimizing the average energy passing through the non-targeted region.

FIG. 9 illustrates that multiple beams from a plurality of emitter arrays with a plurality of different frequencies and pulse “trains” can be used to generate constructive interference increasing the viral inactivation energy for a targeted region while minimizing the average energy passing through the non-targeted region.

In one embodiment, multiple emitters constitute an array of sources. In an integrated version, these amount to a phased array, which can be a commercially available product. In a distributed version, an effective phased array is built from the individual versions. In both cases, the plurality of sources is used to localize the effect to a predetermined location, volume or area. The plurality or sources that directs the electromagnetic field can be arranged in a pattern to increase the peak intensity, for example through constructive interference. It can also be arranged to decrease the peak intensity at a specific location, for example through destructive interference, to protect areas that should not be exposed. The intensity can also be modulated in other ways, for example through a combination of constructive and destructive interference.

The collection of emitters, sources or antennae is controlled via individually controlling timing of pulses, the power, and phase of the microwave signal at each source. It requires a control unit that can be a manual adjustment interface, a microcontroller unit (MCU), a computer, an FPGA, ASIC, or other electronic device.

In summary, the method described herein uses one or several emitters of controlled microwave generation to inactivate one or more viruses through the resonant energy transfer to the virus' acoustic vibrations. The emitters can be controlled in terms of frequency, power, pulse width, location, direction, or phase to suit the application. A control mechanism is present that determine the spatiotemporal distribution of the microwave radiation. The control mechanism can rely on manual inputs, scheduling, sensor signals, feedback algorithms, or other algorithmic approaches, and relies on manual controls, sensors, and/or digital circuits such as processors or ASICs.

Below we describe embodiments of devices that implements the above method.

The device describe herein consists of a power source or connection to a power source, a power processing unit that converts the input power into power for microwave generation, one or more microwave sources, one or more emitters or antennae, and a control system.

FIGS. 1, 3, and 4 shows examples of such devices.

The power connection is a terminal block or a power cable that connects an external power source. The power source can be the grid (110 V AC, 220 VAC or a 3-phase source, ideally via a power socker), a battery, a solar panel, a fuel cell, a generator, a receiver of a wireless power transfer system, or a combination thereof.

The power processing unit converts the input power to a useful form for driving the microwave emitter(s) or source(s). In various embodiments, it converts alternating current (AC) from the electricity source to direct current (DC), it converts direct current (DC) from the electricity source to an alternating current (AC), it converts the electricity from the electricity source to a high voltage, or it does more than one of these steps. In various embodiments, it uses a resonant topology for power conversion. In one embodiment, the power conversion step uses a transformer, with one or more secondary windings to deliver multiple voltages.

In one embodiment, the power processing unit generates a plurality of voltage pulse(s) or plurality of current pulse(s) in some embodiments, and in others a combination of both.

In an embodiment of a pulsed system, the unit incorporates a high voltage capacitor that can store and release sufficient energy for a high peak pulse intensity, for example greater than 1 kW.

In one embodiment, the power processing unit can multiplex between several sources of time-varying electromagnetic fields, e.g., microwave or pulse sources. The one or more microwave source(s) are a magnetron, a Blumlein generator, a klystron, a traveling-wave tube (TWT), a gyrotron, a solid-state power amplifier (SSPA), a laser, a solid-state phased array, a pulse transformer, an inductive coil, a solid state switch, a vacuum tube, a pulse-forming network, a capacitive discharge, or a combination thereof. Variations of the components above are possible. For example, a single magnetron can have a single electron source with multiple cavities operating at two or more frequencies. In a particular example, an array of two or more magnetrons are powered by a single power processing unit. They can also be controlled by a single controlling device.

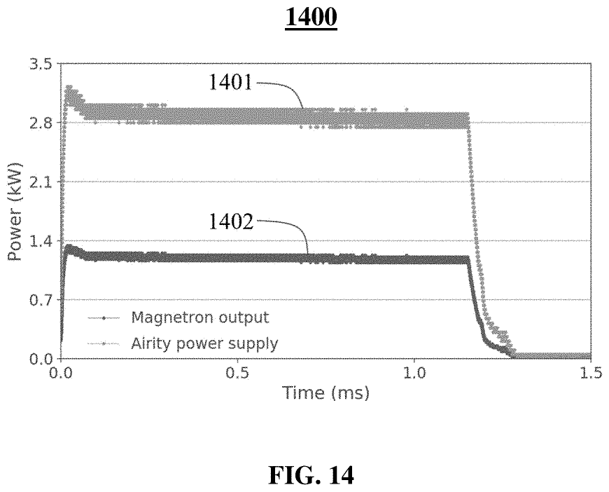

FIG. 14 shows the waveform for our device we made, which is shown in FIG. 3. In the device, a custom high-performance high-voltage power processing unit drives a 9.4 GHz magnetron. The device is operated in pulsed mode. The trace 1401 represents the output power of the power processing unit and the trace 1402 represents the corresponding microwave output power of the magnetron. In this example, the pulse duration is long, larger than 1 ms, which is hundreds of times longer than typical pulsed vacuum electronics systems. The power processing unit demonstrates significant advantages compared with traditional power processing units, or storage of energy in capacitor banks.

The frequency of the microwave sources may overlap with commonly used bands, for which off-the-shelf components are readily available. For example, the SSPA, magnetron or TWT models may operate in the existing microwave communication point-to-point 8 GHz band. They can also operate in the existing 5-6 GHz microwave band, which is close to the expected 6.5 GHz resonant frequency for SARS-CoV-2, which causes COVID 19. A third common band that can be leveraged is the 9-10 GHz microwave band for marine radar. This band is close to the 8.4 GHz frequency for Influenza viruses.

The source(s) of a time varying electromagnetic field, such as microwaves, are connected to one or more emitters, as described above, such as an antenna, a waveguide, a phase array, a transmission line, a reflective surface, a parabolic dish, a dish of a different geometry, a symmetric parabola, a spoiled parabola, a slot antenna, a horn antenna, or a combination thereof.

A directional microwave emitter array can be constructed, composed of two or more devices, or one device with multiple sources and emitters operating at different or identical frequencies, to achieve constructive interference in a focused area. The placement, orientation, and beam forming of the individual emitters can be leveraged to increase the field in the target area, to decrease emission in unwanted areas, and to scan the resulting field across a large area.

The control unit is an integral part of the system and ensures that the emission is not a continuous wave over a long period. Instead, it controls and adjusts the power, direction, pulsing characteristics, etc., to optimize performance and efficiency, while ensuring safety and minimizing off-target radiation.

In a basic embodiment, the control unit turns the emission on and off and/or adjusts the power level power level of the emitted electromagnetic radiation based on a predetermined spatiotemporal pattern and/or a predetermined schedule.

The control unit generates a plurality of pulses to control the duration of emitted electromagnetic radiation. In some cases, it regulates a voltage and/or a current to set the emitted power by a plurality of pulses to control the duration of emitted electromagnetic radiation by the source(s) of time-varying electromagnetic fields and in certain embodiments, it uses active feedback to limit the total emitted peak and/or average power.

The control unit can generate a pulse train, specified by the user or manufacturer or determined based on feedback or sensors, to deliver an optimized dose of radiation. The control unit coordinates the on- and off states of the source(s) of time-varying electromagnetic fields to limit the maximum heating and/or exposure of the treated objects through spatiotemporal averaging.

To specify and control temporally varying emissions, the control unit incorporates a function generator, a signal generator, a pulse generator, or a combination thereof.

The device can be programmed with varying specific operational intervals, for example, a single pulse per hour to thousands of pulses per second.

There are several means by which the control unit can be set and controlled. One implementation is manually through physical buttons or knobs. A second implementation uses a plurality of analog signals to externally control the device. A third implementation is a digitally controlled unit, which can interface with an electronic device such as a desktop computer, laptop, phone, smartphone, a remote control, or microcontroller. One or more of these implementations may be present in the same unit.

The control unit may connect to a network, such as the internet, and use a plurality of digital interfaces, including, but not limited to, ethernet, Bluetooth, etherCAT, CAN bus, RS232, RS485, TTL, serial, WiFi, and cellular communications such as CDMA, GSM, 3G, EDGE, 4G, 5G, WiMAX.

A highly functional, “smart,” device may be connected to a plurality of sensors that can provide information about the target, the surroundings, the environment (temperature, humidity) and the electromagnetic response from the emission, including reflected signals. The plurality of sensors include an internal thermal sensor, a sensor for the monitoring the temperature of the irradiated target, an antenna, a thermal camera, a camera, a photodiode, a GHz antenna, an infrared imaging sensor, an infrared motion sensor, a microphone, a sensor of mechanical vibrations, such as a piezoelectric sensor, a gyroscope, a LiDAR system, a RADAR system, an ultrasound system, a mechanical latch, an electrical switch, a thermometer, a thermocouple, a thermistor, a chemical sensor, a viral detection system such as a microwave absorption system, a PCR system or an antibody-based sensor, an X-ray system, a biological detection system, such as PCR or a biosensor, a latch indicating whether a door is open or closed, an electrical light switch indicating whether light is turned on or off, or a combination thereof.

In an embodiment, the control unit allows for, or does not allow for, electromagnetic radiation to be emitted based on one or more signals from one of more of the sensors listed above. In other cases, the control unit uses the sensor signals, or a computation based on them, to adjust the power level, the pulse shape, duration, phase, and direction, etc. of the radiation.

To coordinate input communications, outputs, and sensor signals, the device has a plurality (one or more) of processors, such as MCUs or CPUs. In an embodiment, it also incorporates a plurality of memory devices. The device comprises a control algorithm, implemented by the processor or other logic circuitry.

The control unit controls the emission of electromagnetic emission through one or several sources. As such, in one embodiment it has a plurality of outputs for individually controlling the radiation of the plurality of source(s). In some embodiments, it also has a plurality of outputs for controlling a plurality of elements that direct the electric field, such as a motorized holder of the antenna, a gimbal, a phased array, or an antenna that changes orientation or shape based on an input signal.

In another embodiment, multiple sources are multiplexed via a common output.

The control unit can thus multiplex between a plurality of sources to control the time-varying electromagnetic field, i.e., the microwave radiation and the pulsing, power, etc. It may also multiplex between the elements that direct the electric fields, such as moving parts or directional antennae.

Several units can work in consort and control units may have communication interfaces and algorithm to communicate with and coordinate with a plurality of similar or different devices. The control unit can also be a connected device which communicates with a computer, server, or centralized system, that determines the operation schedule or parameters or remotely monitors and/or control the system, i.e., a cloud-connected control unit. It can be connected to an external control system or software, via commercially available wired or wireless communication means, such as LAN, Wi-Fi, Bluetooth. The device can be automated or remotely controlled.

In an embodiment that includes a memory unit or a means of communication, the control unit turns the emission on and off, adjusts the power level, or otherwise sets the spatiotemporal pattern based on a schedule that is determined from algorithms and prior sensor data. For example, the unit can sense the presence of people over time and schedule the emission for when people are not present. The algorithm may include machine learning or artificial intelligence/AI.

The safety of the device can be guaranteed based on a range of techniques and incorporated hardware features. In one embodiment, the device or apparatus comprises a plurality of safety mechanisms to limit or control the emitted electromagnetic radiation.

One safety mechanism consists of monitoring the surface temperature of the irradiated object or person and/or monitoring the emitted power. If the surface of the object is too high, or if the emitted radiation exceeds regulatory or other limits, the power is reduced or the device is turned off.

A second mechanisms is to continuously, regularly, or intermittently track and locate humans in time and/or three-dimensional space using infra-red, microphones, cameras, radar, lidar, ultrasonic, or other imaging or ranging techniques. Based on this information, the device can turn off, reduce the power, or change the pulse or frequency characteristics in the presence of people, or selectively target certain physical spaces or areas for radiation based on the location of humans in the space. It can also target areas based on the absence of people at certain times or in certain locations. In this case it can deliver power levels above ICNIRP, FCC and IEEE thresholds.

There are several applications for the method and device described herein. The key advantages over existing methods of viral inactivation are as such:
Effective from close range to significant distances (0 to 10+m) and across large areas, including entire rooms, buildings, and outdoor spaces
Effective without line-of-sight, including through non-metallic walls, dividers, curtains, etc.
Continuous operation with humans present with no interruptions to mission activities
Effective against viruses in free air and on surfaces
Rapid inactivation speed (seconds, not minutes)
Portable or battery-powered
Cost-effective and simple, with no resupply costs or consumables.
Minimal interaction with or damage to surfaces or materials.
Automation removes the opportunity for human error.
Because the power and/or direction of the pulsed emission are variable, the device can be optimized for specific conditions or environments.

In an embodiment, the device can be mounted to a fixed object for temporary or permanent operation, such as a wall, ceiling, light fixture, or a streetlight in private and public spaces to eliminate or reduce viral transmission. The device can scan the area or volume continuously or intermittently to cover the space with high peak powers to eliminate newly shed viral particles.

In an embodiment, the device has a microwave focusing device that can inactivate viruses from a long distance, such as 5 m, 10 m, 25 m, or 100 m.

In an embodiment, the device is combined with a faraday cage to limit exposure beyond a limited volume, consisting of a high-power electromagnetic source of radiation that would irradiate and inactivate viruses inside the volume of the faraday cage while protecting living creatures or inanimate objects from experiencing levels of radiation outside the faraday cage, such that a meaningful amount of viruses are inactivated. In one embodiment, the apparatus is akin to a microwave oven but with a pulsed microwave source at one or more frequencies that differ from the standard 2.45 GHz, for example 5.9 GHz or 9.4 GHz. In a variation of this embodiment, the faraday cage is an entire room, within which a large number of objects, humans or animals, or a smaller number of large objects, humans or animals are exposed to inactivate viruses. The objects include personal protective equipment, shipping boxes, food, and almost any other object that can fit in the container.

In an embodiment, the apparatus is a contained box or container. In this box or container, medical instruments or other sensitive equipment can be placed to be exposed to the source of electromagnetic radiation to sterilize the instruments in a non-thermal manner. The container can be a compact metal enclosure, like a microwave oven. The contained apparatus can be used for easy decontamination of personal protection equipment, including N95 masks, and medical devices.

In an embodiment, a device can be placed for in-line high-volume viral inactivation of liquids or gases, for example, in drinking water supply piping, sewage treatment plant, or a beverage bottling facility.

In an embodiment, the device has a plurality of sources of electromagnetic radiation that is directionally focused on a specific area. The area is, for example, a conveyor belt such that all non-living objects would either pass through the beam or be scanned by the beam. Objects include packages in a warehouse, mail in a sorting facility, or food in a food processing plant.

In an embodiment, the device can be ceiling mounted and project a “curtain” or “grid” of radiation to create virtual containment zones around a series of patient beds as sufficient replacement for a negative pressure room, such as in a temporary hospital configured in a stadium or gymnasium.

In an embodiment, the device resembles a body scanner which a human or other organism can walk through to receive whole-body surface viral inactivation. This may be done while being simultaneously scanned for security purposes, such as in an airport, train station, stadium, or other public venue.

In an embodiment, the device can inactivate viruses in multiple physical spaces simultaneously, in spite of visual and/or physical barriers between those spaces, such as an office environment with cubicle walls, or drywall partitions. This is particularly effective if the partitioning material does not significantly absorb microwave radiation.

In an embodiment, the device is used to create a containment zone or virtual curtain, around an area with an infected person or animal, to prevent spread of the virus to adjacent areas, people, or healthcare workers. For this, the previous, and other embodiments, the containment zone or the exposed area can be indicated by a light source such as an LED, a plurality of lasers, an audible alarm, or physical barrier.

In an embodiment, the device allows for all viruses to be inactivated over a very large area by using a very high power impulse for a very short duration. The large area is, for example, an entire house, building, or section of a city. The impulse(s) may be controlled directionally to minimize overlap of sequential impulses.

In an embodiment, the device's microwave emissions are combined with UV and/or ozone-based sanitization to effect inactivation of viruses as well as other pathogens, such as bacteria. This can be used in hospitals and care facilities for infection control.

In an embodiment, the apparatus is combined with a thermal or chemical disinfection system.

In an embodiment, the device or its emitter is incorporated into a lighting ballast to generate a wide-area viral inactivation effect, such as in a streetlight, a shop light, or regular lighting fixture in an office or private residence.

In an embodiment, the device is incorporated into an HVAC system to inactivate viruses in circulating air within a building, a cruise ship, a bus, a train, an airplane, a car, or any other construction within which an HVAC system is used. By incorporating the device described herein, inactivation or elimination of viruses can be achieved without requiring filters with pore sizes smaller than 200 nm, 100 nm, 50 nm or less.

In an embodiment, the device can be mounted to a land-based vehicle, such as a robot or a car, truck, bike, motorcycle, scooter, semi-truck, bus, subway train, train or taxi, an aerial vehicle such as an airplane, UAV (unmanned aerial vehicle), helicopter, zeppelin, balloon, missile, projectile, glider, airplane or hang glider, or a space vehicle, such as a low earth orbit satellite or a geo-stationary satellite, or rocket.

In an embodiment, a sufficiently high-power source of electromagnetic radiation is used to inactivate viruses of various types within the confines of a specific container or object, such as a shipping container at a port of entry or other location, such that a meaningful amount of viruses are inactivated.

In an embodiment, the device has an emitter located such that it irradiates a container or pipe containing human or animal waste products, as well as the surrounding area, including but not limited to toilet system. For example, a porta-potty would have an emitter in the ceiling and inside the waste tank, and after every occupant leaves, both the booth and the tank are inactivated of viruses. In a second example, a conventionally flushed toilet has a seat and bowl that are irradiated before, during, or after every flush. In a third example, the pipe exiting the bottom of the toilet would have an emitter pointed at it with sufficient power to inactive viruses as they briefly pass through the beam, such that a meaningful proportion of exposed of viruses are inactivated.

In an embodiment, the device is portable. The portable device may be powered by a battery. The portable device can be handheld, wearable, or mounted on a cart or other small means of attachment.

In an embodiment, the device is attached to or worn by a human to deliver a long-term protection against or treatment for a viral infection.

In an embodiment, at least two directional microwave emitters operating from non-adjacent locations form an array such that the beams create constructive interference in a focused target area, achieving viral inactivation while minimizing human radiation exposure. In a variation, the directional microwave emitter array is composed of two or more devices operating at different frequencies to achieve overlapping effects or constructive interference a focused area.

In an embodiment, the device comprises an array of microwave sources tuned to various frequencies to inactivate specific viruses, or a panel or range of viruses.

In an embodiment, the device comprises a plurality of safety mechanisms to limit the emitted electromagnetic radiation.

### Medical Device

The apparatus described herein can be used to great effect for medical purposes, including the prevention and treatment of infection in human and animal subjects. A medical device based the antiviral properties of microwave energy transfer to acoustic vibrations has several application and multiple variations of such a medical device are described below.

A method of viral inactivation consists of exposing viral particles to acoustic resonance stimulation using non-ionizing gigahertz electromagnetic radiation, wherein the viruses are present in infected humans, including patients with COVID-19, or animals, including pets or livestock. It can result in clinically significant positive outcomes for humans and other animals, including but not limited to an accelerated speed of recovery. The treatment may reduce the risk of advancing to severe disease, including hospitalization and the development of complications including pneumonia, organ injury and/or failure, secondary infection, sepsis, septic shock, and/or acute respiratory distress syndrome and acute respiratory failure.

The method can reduce the viral load by about >99.999%, about >99.99%, about >99.9%, about >99%, about >95%, about >90%, about >80%, about >70%, about >60%, about >50%, about >40%, about >30%, or about >20%, depending on the virus, the location of infection, the treatment protocol, etc.

As a consequence, the method reduces transmission of the virus from its source or tissue of origin or initial infection (e.g., the respiratory system) to other tissues, organs or systems. It also reduces the risk of viral transmission to other humans, including family members and healthcare workers. Treatment can also reduce the risk of viral transmission to other animals and from animals to humans.

In principle, nearly all human viruses (see list above) can be prevented or treated by the right combination of power, frequency, and timing of the emitted radiation, provided that the infection or the treated tissue or fluid is not significantly larger than the skin depth for the radiation at the prescribed frequency. The device can therefore be used to inactivate viruses on the surface of the skin of living creatures to a certain depth, for example, greater than 1 mm, and less than 2 cm.

Prevention or treatment of internal organs can also be achieved through a method wherein the emitter or antenna is inserted into a body orifice, such as the mouth, throat, anus, vagina, or nose to deliver therapeutic levels of radiation to the target organ, such as the lungs, stomach, heart, liver, spleen, bones, blood, pancreas, etc. Because the microwaves can penetrate through tissue (albeit with some attenuation), proximity to the target region may be sufficient such that the larger organs may not have to be injected directly.

One example is a flexible metalized waveguide that can be used to emit radiation from inside the target subject after being inserted through an orifice. One such example could be a ventilation tube that has a mylar tape applied to it to create an antenna that would efficiently inactivate viruses in the lungs of a human patient.

For more direct targeting, there is a waveguide catheter that can be inserted into blood vessels which enables the use of lower peak pulse power to achieve therapeutic levels for interior tissues and organs, including the heart, lungs, and muscle tissues.

For the treatment of human subjects, the dosing and safety is paramount. In an embodiment, the device can detect and measure the tissue thickness of the patient and vary peak power outputs to achieve sufficient viral inactivation depth. In one example, the tissue thickness of the patient can be measured by detecting the amount of microwave energy absorbed as heat using ultrasound imaging.

The dose can also be adjusted or determined based on detection of the patient temperature. For surface treatments, detection can be performed by monitoring the surface temperature of patients. For internal treatments, the internal temperature may be monitored. In both cases, monitoring is performed with the goal of limiting the temperatures increase to a within a safe range, for example 1° C. above normal, while maximizing the effect through adjustment of peak power, frequency, pulse width, repetition frequency, etc.

To reduce off-target effects, the microwave radiation beam can be focused to minimize undesired radiation of human patients or specific living tissues. The pulse width of the microwave emission can also be controlled based on the size of the area to be inactivated as well as the emitter distance to the patient. The device can have a means of tracking the position of the patient relative to the emitters and use active feedback to ensure that the beams stay focused on the target volume. In more advanced models, the structure of the tissues is modeled, or directly measured by guide beams, such that active beam forming can be implemented. The method is analogous to a guide star and adaptive optics in microscope or telescopes.

Better targeting is also achieved using a microwave emitter array, or a movable source, which directs a sequence of microwave beams at a target volume, e.g., a section of the lung, from multiple angles, with the patient positioned so the target volume is at the center of rotation (isocenter) of the beams. As the angle of the radiation source changes by moving the emitter(s) around the patient, every beam passes through the target volume but passes through a different area of tissue on its way to and from the target volume. As a result, the cumulative radiation dose at the target volume is relatively high, and the average radiation dose to non-targeted tissue is relatively low. The beams can also be emitted simultaneously for a higher power in the overlapping volume, compared with adjacent areas (not including attenuation).

Treatment and prevention of viral infection can be achieved outside of the patient. In certain cases, the treatment can readily be integrated into existing types of medical devices, such as ventilators, oxygen therapy, C-PAP machines, dialysis machines, intravenous delivery systems, whereby existing intubations or catheters can be used for viral inactivation in addition to transferring air, gases, or body fluids. In an embodiment, the device encapsulates catheters, gastric tubes, lung intubations, and other medical conduits to perform inline inactivation of viruses. In a second embodiment, the device inactivates viruses in blood by being placed in series with, or within, a blood transfusion system.

Active measurement of the viral load can be used to determine the effect of the treatment and to calculate the original dose. In one embodiment, the device can measure the viral load of a patient by detecting the relative absorption of microwave energy.

In a second embodiment the device can detect the presence of viruses in a patient via a tissue, mucosa, or other body fluid sample, and/or diagnose the specific types of virus present, and deliver either a single pulse or series of pulses specifically targeting and inactivating those viruses types detected in the patient sample.

In another implementation, the device is combined with a viral detection or quantification system that can determine the degree of inactivation after operation, or that can continuously monitor viral levels during operation until the viral load is gone or has reached an acceptable limit. One example is continuous monitoring of patient blood during dialysis. Another is the measurement of viral load in sputum or saliva samples using an antibody or PCR assay following treatment of the airways.

For ongoing treatment with intermittent treatment intervals, or in situations where many patients need treatment or are nearby, the device can detect the elevated temperature of human patients indicating a fever, and selectively deliver radiation to those specific patients.

The method of viral inactivation as described herein can be used in combination with another medical procedure, such as bronchoscopy, colonoscopy, endoscopy, sigmoidoscopy, gastroscopy. In these procedures, a scope is inserted into the patient already, such that antiviral treatment of internal organs or cavities may be performed without a significant increase in risk, time, and cost to the patient.

What is claimed is:

1. A method for selectively inactivating viruses comprising:
generating non-ionizing gigahertz electromagnetic radiation at a frequency that stimulates an acoustic resonance within a virus;
radiating the electromagnetic radiation onto a predetermined area or into a predetermined volume; and
controlling the temporal characteristics of the radiation by modulation of the transmitted power

2. The method of claim 1, wherein the radiation is pulsed to achieve high peak fields that inactivate the virus while limiting the average exposure of the target area or volume.

3. The method of claim 1, wherein the radiation is radiated from an antenna or phased array and wherein the beam is scanned spatially to cover an area or volume larger than the width or size of the beam.

4. The method of claim 1, wherein the electromagnetic radiation is emitted in a plurality of frequencies to improve the viral inactivation and wherein the two or more frequencies are emitted simultaneously, sequentially, or a combination thereof.

5. The method of claim 1, wherein the radiation intensity is modulated within or between pulses.

6. The method of claim 1, wherein the radiation is emitted from a plurality of sources to increase the viral inactivation.

7. The method of claim 1, wherein the method is used in combination with a second antimicrobial or antiviral method.

8. The method of claim 1, wherein the target area or volume is pretreated to maximize the antiviral effect.

9. The method of claim 1, wherein the target object is a human patient.

10. The method of claim 1, wherein the radiation source is places on a wall, ceiling, floor, or mounting post to irradiate an area or volume, such as a room, hallway, building, outdoor space, or vehicle interior.

11. The method of claim 1, wherein the radiation source is placed in or on a movable object, such as a robot, ground vehicle, aerial vehicle, or marine vessel, to deliver the antiviral electromagnetic radiation to a target area.

12. An apparatus for inactivating viruses by using the method according to claim 1, comprising:
a power processing unit takes input power and drives a microwave source;
one or more microwave sources;
one or more antennae or emitters; and
a control unit that modulates the radiation spatially or temporally.

13. The apparatus in claim 12, wherein the apparatus incorporates a plurality of sensors and wherein the control unit uses sensor data to modulate the radiated beam.

14. The apparatus in claim 12, wherein the control module has a one or more user interfaces by which the operation can be determined, adjusted, or monitored.

15. The apparatus in claim 12, wherein the control module is connected to a network.

16. The apparatus in claim 12, wherein the radiation is confined to a container where objects or people are treated for inactivating viruses.

17. The apparatus in claim 12, wherein the apparatus is portable.

18. The apparatus in claim 12, where in the apparatus comprises:
a pulsed high voltage power supply;
a magnetron;
a microwave horn or slotted antenna; and
a control module

19. The apparatus in claim 12, wherein the apparatus is used to treat or prevent a viral infection in a patient.

20. The apparatus in claim 12, wherein the apparatus is exposing tissues or bodily fluids with radiation at a frequency matches the acoustic resonant frequency of the targeted virus.
